(19) United States
(12) Patent Application Publication
(10) Pub. No.: US 2025/0179577 A1
(43) Pub. Date: Jun. 5, 2025

(54) SCREENING METHOD OF AUTOLYSOSOME GENE C10ORF10 FOR REGULATING ADIPOSE FUNCTION OF OBESE PATIENTS

(71) Applicant: THE AFFILIATED HOSPITAL OF QINGDAO UNIVERSITY, Qingdao (CN)

(72) Inventors: Yujie Deng, Qingdao (CN); Ping Wang, Qingdao (CN); Qing Yu, Qingdao (CN); Yajun Jing, Qingdao (CN); Xiaoxia Wang, Qingdao (CN); Shumin Xu, Qingdao (CN)

(21) Appl. No.: 18/886,841

(22) Filed: Sep. 16, 2024

(30) Foreign Application Priority Data
Dec. 4, 2023 (CN) 2023116513069

(51) Int. Cl. C12Q 1/6883; C12Q 1/6874

(52) U.S. Cl. CPC C12Q 1/6883; C12Q 1/6874; C12Q 2600/158

(57) ABSTRACT

A screening method of an autolysosome gene C10ORF10 for regulating fat function of obese patients is provided, including collecting subcutaneous and visceral adipose tissues from people with different metabolic conditions and different BMIs, followed by transcriptome sequencing analysis; dividing specimens from people with normal metabolism into two groups of BMI<25 kg/m² and BMI>30 kg/m², comparing the two groups in terms of expression differences of autolysosome-related genes in subcutaneous and visceral adipose tissues, screening out differential genes; analyzing and observing several autolysosome-related genes in terms of differential expressions existed in the subcutaneous and visceral adipose tissues of the two groups of BMI<25 kg/m² and BMI>30 kg/m², where a gene with a greatest expression difference is C10ORF10.

### CROSS-REFERENCE TO RELATED APPLICATIONS

This application claims priority to Chinese Patent Application No. 2023116513069, filed on Dec. 4, 2023, the contents of which are hereby incorporated by reference for all purposes.

### TECHNICAL FIELD

The present application relates to the technical field of gene screening, and in particular to a screening method of an autolysosome gene C10ORF10 for regulating fat function of obese patients.

### BACKGROUND

Obesity, with a prevalence increasing every year, is closely associated with the occurrence of hyperglycemia, hypertension, hypertriglyceridemia, hypo high-density lipoprotein (HDL) cholesterolemia and hyperuricemia. Adipose tissue dysfunction has been identified as a key factor in the progression of obesity-related complications, and a large number of studies have found that autolysosomes plays an important role in the regulation of adipose function. Therefore, a feasible method capable of screening autolysosome genes that regulate adipose functions in obese patients is crucial for adiposity, and a screening method for the autolysosome gene C10ORF10 that regulates adipose function in obese patients is thus proposed.

### SUMMARY

It is an objective of the present application to address the problems existing in the prior art. To achieve the above objectives, the present application provides following technical schemes: a screening method of an autolysosome gene C10ORF10 for regulating fat function of obese patients, including following steps:
step 1, determining autolysosome genes firstly, and collecting subcutaneous and visceral adipose tissues from people with different metabolic conditions and different body mass indexes (BMIs), followed by transcriptome sequencing analysis;
step 2, dividing specimens collected from people with normal metabolism into two groups of BMI<25 (kilograms per cubic meters, kg/m²) and BMI>30 (kg/m²), comparing the two groups in terms of expression differences of autolysosome-related genes in subcutaneous and visceral adipose tissues, screening out differential genes, followed by comparison of expressions of the differential genes in adipose tissues of the two groups in systems of Genecards, The Human Protein Atlas, and the National Center for Biotechnology Information, selecting differential genes with high expression for analysis of a following step; and
step 3, analyzing and observing the subcutaneous and visceral adipose tissues, comparing several autolysosome-related genes in terms of differential expressions existed in the two groups of BMI<25 (kg/m²) and BMI>30 (kg/m²), where a gene with a greatest expression difference is C10ORF10.

A preferred technical scheme of the present application includes: step 4, carrying out correlation analysis of a differential gene C10ORF10 with BMI in subcutaneous and visceral adipose tissues of people with different BMIs, where expression differences of C10ORF10 mRNA in adipose tissues of patients with different BMIs and correlation with BMI are firstly observed, and highly expressed C10ORF10 mRNA is observed in subcutaneous adipose tissues of patients with a BMI of >30 (kg/m²) according to collected and analyzed RNA-seq results of human adipose tissues, suggesting a significant correlation with BMI.

A preferred technical scheme of the present application includes: step 5, conducting correlation analysis of differential genes with blood glucose, blood lipids, blood pressure and blood uric acid in obese patients with different metabolic conditions, including high uric acid, high blood lipids, high blood glucose, and high blood pressure; correlations of baseline C10ORF10 mRNA with baseline and post-follow-up reductions in blood glucose and HbA1c are observed in patients undergoing bariatric surgery, where C10ORF10 mRNA levels are observed to be positively correlated not only with postprandial glucose and HbA1c of baseline, but also with decreases in both postprandial glucose and HbA1c of post-follow-up, indicating that C10ORF10 mRNA is also involved in glucose regulation before and after weight-loss surgery in obese patients.

A preferred technical scheme of the present application includes: step 6, constructing mice with conditional knockout or overexpression of the differential gene C10ORF10 in adipose tissue and performing related studies, including overall phenotypic observations, adiposity content and morphological observations in mice, adipogenic differentiation and tissue development, lipolysis, adipose tissue inflammation and overall inflammation, browning of subcutaneous fat, ectopic deposition of lipids in liver and skeletal muscles.

In a preferred technical scheme of the present application, specific steps of determining autolysosome genes in the step 1 include:
S101, determining autolysosome genes, and identifying a list of autolysosome-related genes through literature research and database querying;
S102, collecting samples, whereby obese patients with different metabolic conditions and different BMIs are selected as study objects and samples are collected from subcutaneous and visceral adipose tissues of the study objects, with a collection process following ethical regulations and relevant research ethics approvals obtained; and
S103, extracting total RNA, where total RNA is extracted from subcutaneous and visceral adipose tissue samples using a method of RNA extraction kit according to instructions, and high quality of RNA is ensured.

In a preferred technical scheme of the present application, specific steps of collecting subcutaneous and visceral adipose tissues from people with different metabolic conditions and different BMIs, followed by transcriptome sequencing analysis in the step 1 include:
step a, preparing a library, where an RNA sample is used for library preparation according to a transcriptome sequencing methodology, typically including steps of RNA reverse transcription, cDNA synthesis, and library construction;
step b, sequencing, where the library is subjected to high-throughput sequencing, either by an RNA-seq method for transcriptome sequencing, or by sequencing using an Illumina platform; and
step c, analyzing data, where the data obtained from sequencing are subjected to pre-processing steps of quality control, removal of low-quality data and filtering of low-expressed genes, followed by comparative, quantitative and differential expression analyses of transcriptome data using bioinformatics analysis tools.

In a preferred technical scheme of the present application, specific steps of analyzing and observing the subcutaneous and visceral adipose tissues, comparing several autolysosome-related genes in terms of differential expressions existed in the two groups of BMI<25 (kg/m²) and BMI>30 (kg/m²) in the step 3 include:
S301, analyzing differential genes, where the expression differences of autolysosome-related genes in subcutaneous and visceral adipose tissues of the two groups of BMI<25 (kg/m²) and BMI>30 (kg/m²) are compared based on results of transcriptome sequencing analyses in the step 2, and a statistical method is available to conduct differential gene analyses;
S302, determining the gene with the greatest expression difference, where the gene with the largest expression difference is identified from the differential genes, and the autolysosome-related genes with a most significant expression difference from samples of the two groups are screened out based on results of statistical analyses;
S303, verifying the genes with the greatest expression difference, where the gene with the largest expression difference is validated using a method of real-time quantitative polymerase chain reaction (qPCR), and the expression difference of the gene in the samples of the two groups is further confirmed by quantitative analysis of gene expression levels; and
S304, conducting database querying and literature research, where public databases of systems of Genecards, The Human ProteinAtlas and National Center for Biotechnology Information are used to query relevant information about the C10ORF10 gene, and to obtain a mechanism of the gene in a function of the autolysosome.

In a preferred technical scheme of the present application, specific steps of carrying out correlation analysis of a differential gene C10ORF10 with BMI in subcutaneous and visceral adipose tissues of people with different BMIs in the step 4 include:
S401, preparing data, where expression data of the C10ORF10 gene and corresponding BMI data in the samples selected are collated;
S402, processing data, where the samples are divided into different BMI groups based on the BMI data of the samples, such as low BMI group and high BMI group, while the expression data of the C10ORF10 gene is matched with the BMI data;
a preferred technical scheme of the present application also includes: S403, conducting a correlation analysis, where statistical methods, such as Pearson correlation coefficient and Spearman correlation coefficient, are used to conduct correlation analyses of C10ORF10 gene expression and BMI, and a correlation coefficient, strength of correlation, and direction of correlation between expression levels of the C10ORF10 gene and BMI are calculated;
a preferred technical scheme of the present application also includes: S404, conducting statistical analyses, where results of the correlation analyses are used to conduct statistical tests to determine if a significant correlation exists between the C10ORF10 gene expression and BMI, followed by statistical tests using t-tests and analysis of variance.

Compared with the prior art, the present application has the beneficial effects that:
according to the present application, subcutaneous fat and visceral fat tissues of people with different metabolic conditions and BMIs are collected and subjected to transcriptome sequencing analysis, then the samples of people with normal metabolism are divided into two groups of BMI<25 (kg/m²) and BMI>30 (kg/m²), followed by comparing the expression differences of autolysosome-related genes in subcutaneous and visceral adipose tissues of the two groups; then the differential genes are screened and the expression of the screened differential genes in adipose tissue is compared in the websites of Genecards, The Human ProteinAtlas, and National Center for Biotechnology Information, and the differential genes with high expression are selected for analysis and found that there is differential expression of several autolysosome-related genes in subcutaneous and visceral adipose tissue in the two groups of BMI<25 (kg/m²) and BMI>30 (kg/m²), and the gene with the greatest expression difference is C10ORF10, suggesting that the autolysosome gene that regulates the function of adipose tissue in obese patients is effectively screened out.

### BRIEF DESCRIPTION OF THE DRAWINGS

FIG. 1A is an expression diagram of autolysosome-related genes in subcutaneous of patients with different body mass indexes (BMIs).

FIG. 1B is an expression diagram of autolysosome-related genes in visceral fat of patients with different BMIs.

FIG. 2 shows expression of C10ORF10 in different cells.

FIG. 3A shows C10ORF10 expression in subcutaneous fat in people with different BMIs.

FIG. 3B shows C10ORF10 expression in visceral fat in people with different BMIs.

FIG. 4A shows correlation of C10ORF10 mRNA in subcutaneous adipose tissue with BMI.

FIG. 4B shows correlation of C10ORF10 mRNA in visceral adipose tissue with BMI.

FIG. 5A shows correlation of baseline subcutaneous fat C10ORF10 with baseline glucose in patients underwent bariatric surgery.

FIG. 5B shows correlation of baseline subcutaneous fat C10ORF10 with baseline glycated haemoglobin in patients underwent bariatric surgery.

FIG. 5C shows correlation between baseline subcutaneous fat C10ORF10 and postprandial glucose reduction in patients after bariatric surgery.

FIG. 5D shows correlation of baseline subcutaneous fat C10ORF10 with glycated haemoglobin reduction in patients after bariatric surgery.

FIG. 6 shows a process of a screening method of an autolysosome gene C10ORF10 for regulating fat function of obese patients provided by the present application.

FIG. 7 shows specific steps of determining autolysosome genes in a step 1 in the screening method provided by the present application.

FIG. 8 shows specific steps of after determining autolysosome genes in the step 1 of the screening method provided by the present application.

FIG. 9 shows specific steps of a step 3 in the screening method provided by the present application.

FIG. 10 shows specific steps of a step 4 in the screening method provided by the present application.

### DETAILED DESCRIPTION OF THE EMBODIMENTS

To make the objectives, technical schemes and advantages of the embodiments of the present application clearer, the technical schemes in the embodiments of the present application are described clearly and completely in the following, in conjunction with the accompanying drawings.

Therefore, the following detailed description of the embodiments of the present application is not intended to limit the scope of the present application for which protection is claimed, but represents only some embodiments of the present application. Based on the embodiments in the present application, all other embodiments obtained by a person of ordinary skill in the art without creative labor fall within the scope of protection of the present application, and it is to be noted that the embodiments and the features and technical schemes in the embodiments in the present application may be combined with each other in the absence of conflict, and it is to be noted that: similar symbols and letters denote similar items in the following accompanying drawings, and thus once an item is defined in one of the accompanying drawings, there is no need to further define and explain it in the subsequent accompanying drawings.

### Embodiment 1

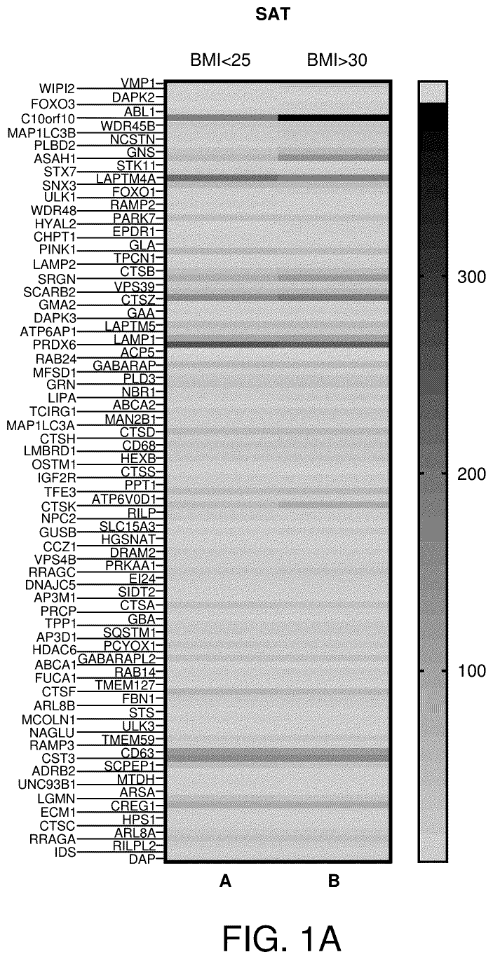

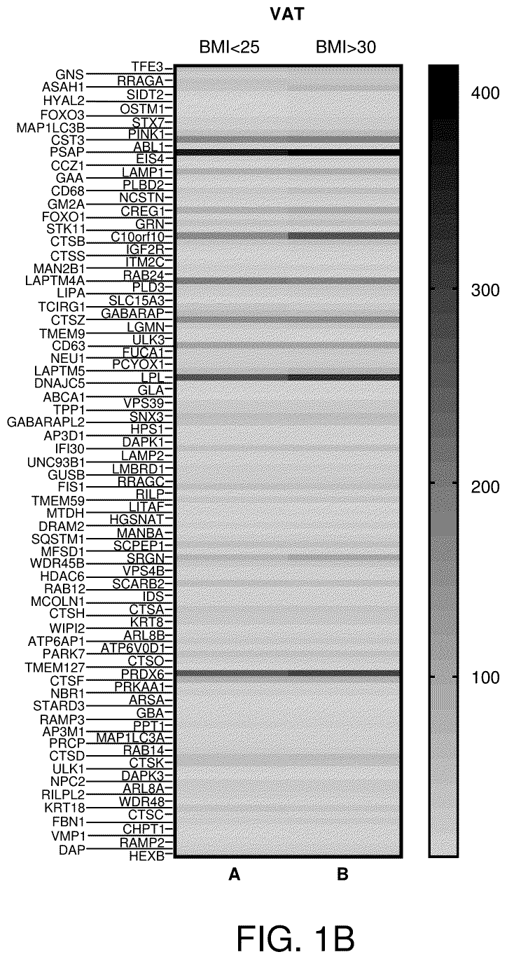

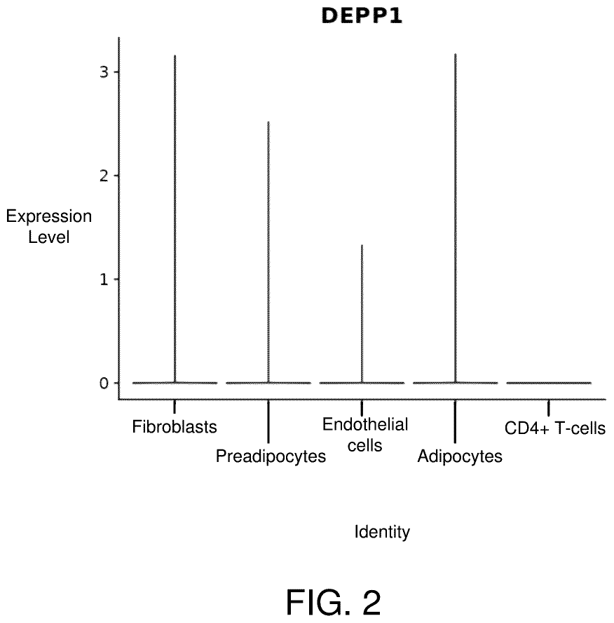

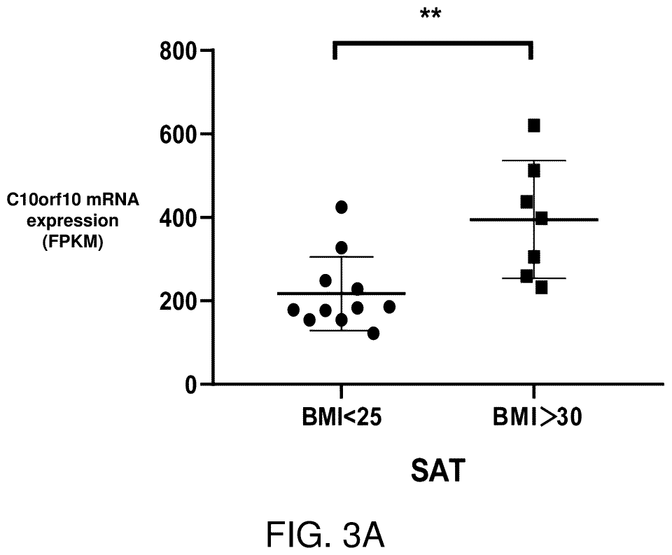

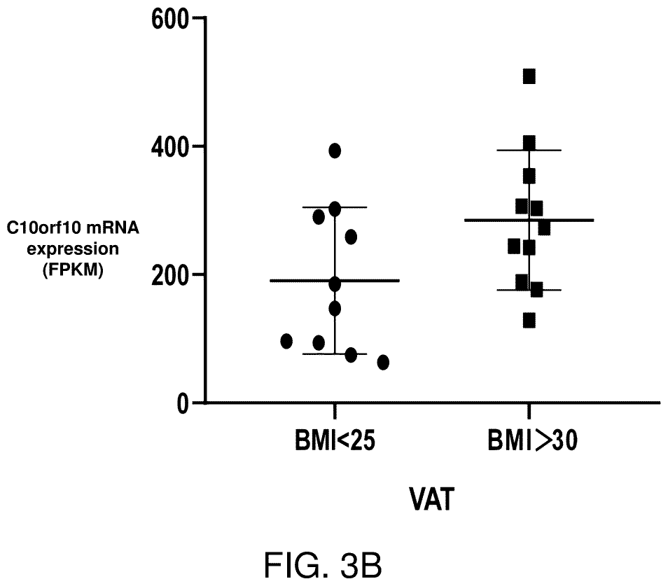

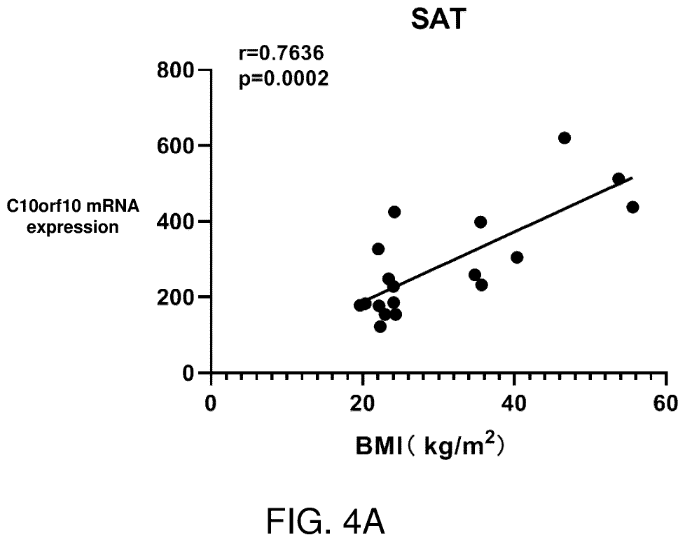

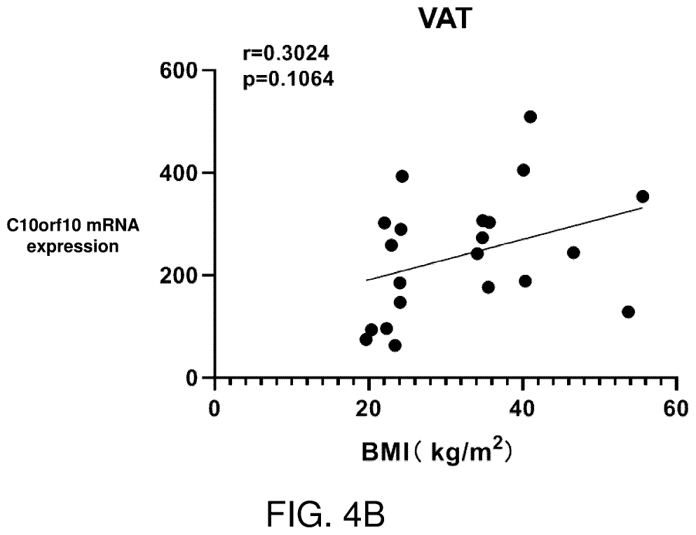

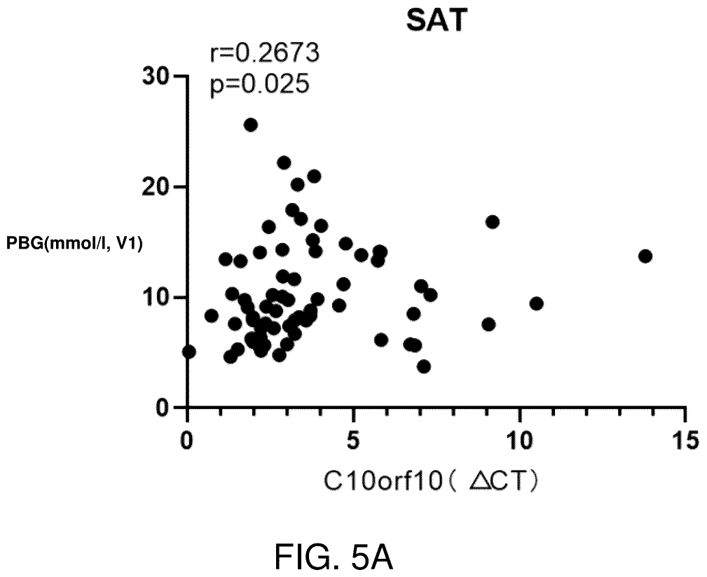

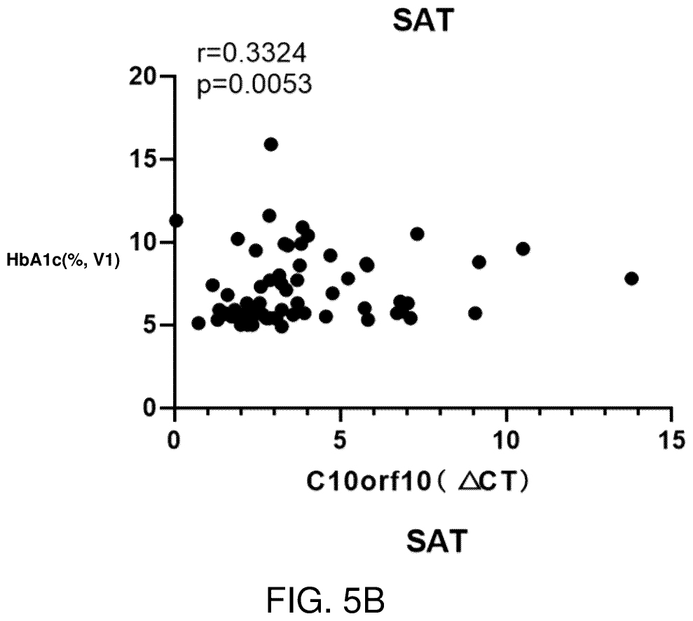

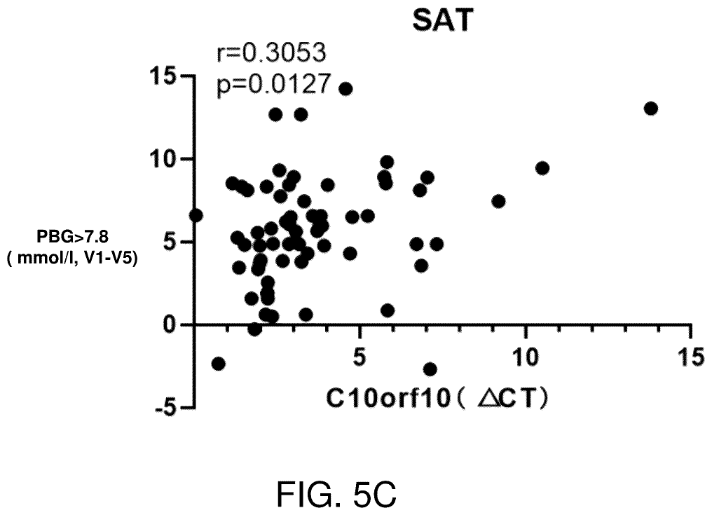

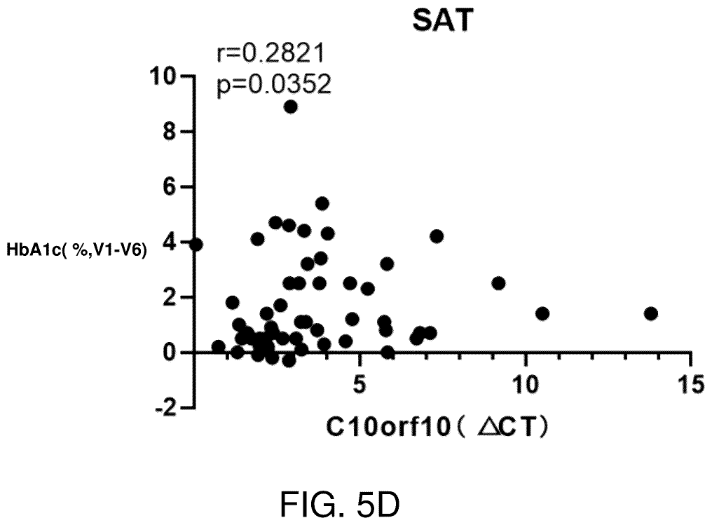

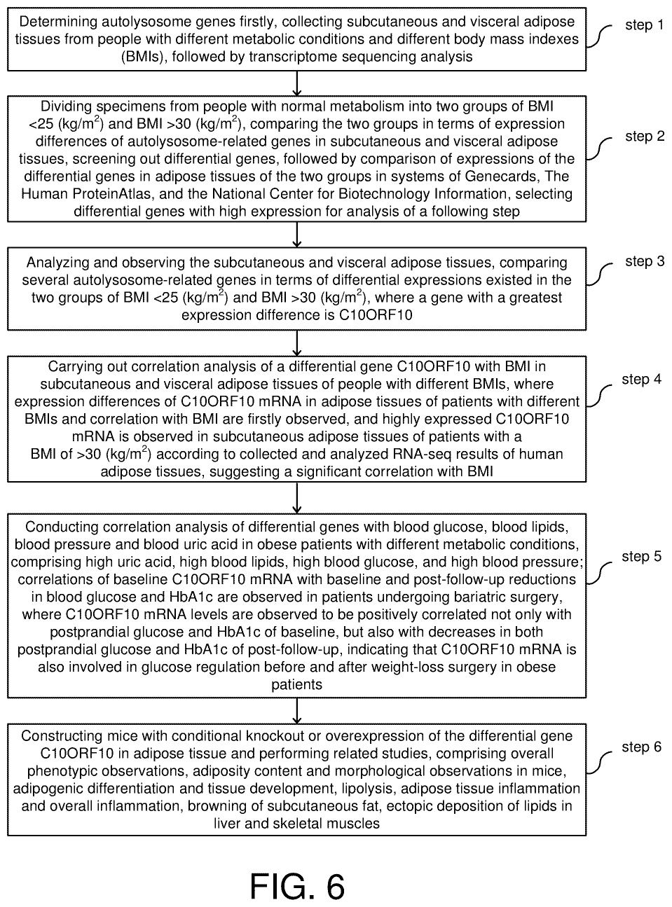

Referring to FIG. 1A, FIG. 1B, FIG. 2, FIG. 3A, FIG. 3B, FIG. 4A, FIG. 4B, and FIG. 5A-FIG. 5D, the present application provides a screening method of an autolysosome gene C10ORF10 for regulating the fat function of obese patients, including following steps as shown in FIG. 6:
step 1, determining autolysosome genes firstly, collecting subcutaneous and visceral adipose tissues from people with different metabolic conditions and different body mass indexes (BMIs), followed by transcriptome sequencing analysis;
step 2, dividing specimens from people with normal metabolism into two groups of BMI<25 (kg/m²) and BMI>30 (kg/m²), comparing the two groups in terms of expression differences of autolysosome-related genes in subcutaneous and visceral adipose tissues, screening out differential genes, followed by comparison of expressions of the differential genes in adipose tissues of the two groups in systems of Genecards, The Human ProteinAtlas, and the National Center for Biotechnology Information, selecting differential genes with high expression for analysis of a following step;
step 3, analyzing and observing the subcutaneous and visceral adipose tissues, comparing several autolysosome-related genes in terms of differential expressions existed in the two groups of BMI<25 (kg/m²) and BMI>30 (kg/m²), where a gene with a greatest expression difference is C10ORF10;
step 4, carrying out correlation analysis of a differential gene C10ORF10 with BMI in subcutaneous and visceral adipose tissues of people with different BMIs, where expression differences of C10ORF10 mRNA in adipose tissues of patients with different BMIs and correlation with BMI are firstly observed, and highly expressed C10ORF10 mRNA is observed in subcutaneous adipose tissues of patients with a BMI of >30 (kg/m²) according to collected and analyzed RNA-seq results of human adipose tissues, suggesting a significant correlation with BMI;
step 5, conducting correlation analysis of differential genes with blood glucose, blood lipids, blood pressure and blood uric acid in obese patients with different metabolic conditions, including high uric acid, high blood lipids, high blood glucose, and high blood pressure; correlations of baseline C10ORF10 mRNA with baseline and post-follow-up reductions in blood glucose and HbA1c are observed in patients undergoing bariatric surgery, where C10ORF10 mRNA levels are observed to be positively correlated not only with postprandial glucose and HbA1c of baseline, but also with decreases in both postprandial glucose and HbA1c of post-follow-up, indicating that C10ORF10 mRNA is also involved in glucose regulation before and after weight-loss surgery in obese patients; and
step 6, constructing mice with conditional knockout or overexpression of the differential gene C10ORF10 in adipose tissue and performing related studies, including overall phenotypic observations, adiposity content and morphological observations in mice, adipogenic differentiation and tissue development, lipolysis, adipose tissue inflammation and overall inflammation, browning of subcutaneous fat, ectopic deposition of lipids in liver and skeletal muscles.

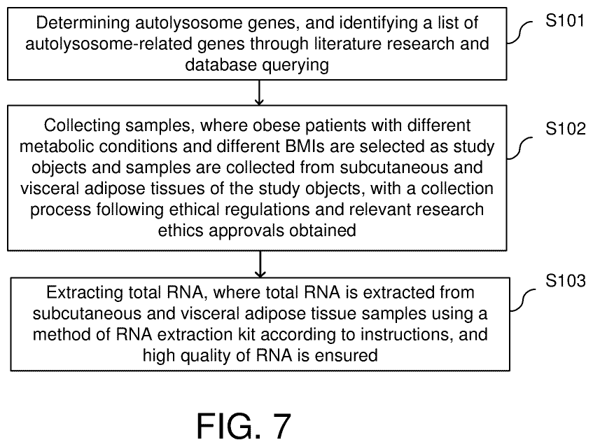

As shown in FIG. 7, specific steps of determining autolysosome genes in step 1 include:
S101, determining autolysosome genes, and identifying a list of autolysosome-related genes through literature research and database querying;
S102, collecting samples, whereby obese patients with different metabolic conditions and different BMIs are selected as study objects and samples are collected from subcutaneous and visceral adipose tissues of the study objects, with a collection process following ethical regulations and relevant research ethics approvals obtained; and
S103, extracting total RNA, where total RNA is extracted from subcutaneous and visceral adipose tissue samples using a method of RNA extraction kit according to instructions, and high quality of RNA is ensured.

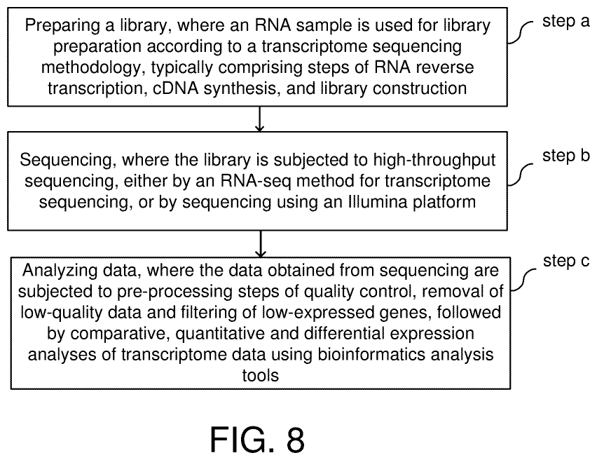

As shown in FIG. 8, specific steps of collecting subcutaneous and visceral adipose tissues from people with different metabolic conditions and different BMIs, followed by transcriptome sequencing analysis in step 1 include:
step a, preparing a library, where an RNA sample is used for library preparation according to a transcriptome sequencing methodology, typically including steps of RNA reverse transcription, cDNA synthesis, and library construction;
step b, sequencing, where the library is subjected to high-throughput sequencing, either by an RNA-seq method for transcriptome sequencing, or by sequencing using an Illumina platform; and
step c, analyzing data, where the data obtained from sequencing are subjected to pre-processing steps of quality control, removal of low-quality data and filtering of low-expressed genes, followed by comparative, quantitative and differential expression analyses of transcriptome data using bioinformatics analysis tools.

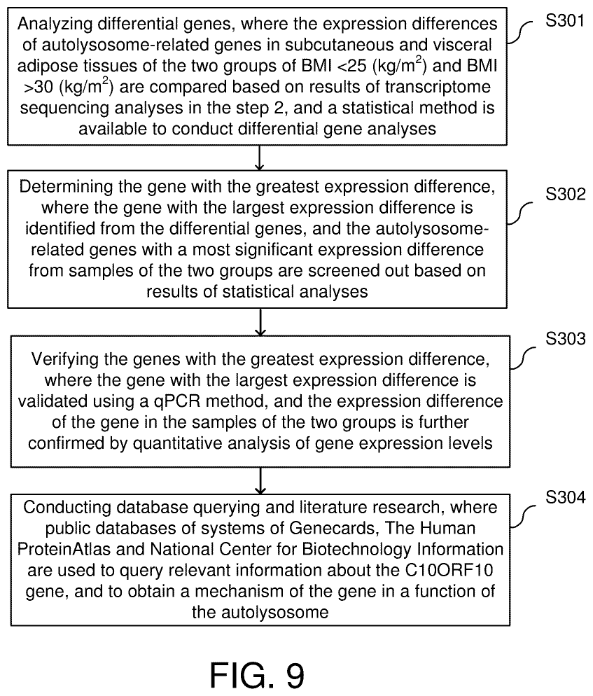

As shown in FIG. 9, specific steps of analyzing and observing the subcutaneous and visceral adipose tissues, comparing several autolysosome-related genes in terms of differential expressions existed in the two groups of BMI<25 (kg/m²) and BMI>30 (kg/m²) in the step 3 include:
S301, analyzing differential genes, where the expression differences of autolysosome-related genes in subcutaneous and visceral adipose tissues of the two groups of BMI<25 (kg/m²) and BMI>30 (kg/m²) are compared based on results of transcriptome sequencing analyses in the step 2, and a statistical method is available to conduct differential gene analyses;
S302, determining the gene with the greatest expression difference, where the gene with the largest expression difference is identified from the differential genes, and the autolysosome-related genes with a most significant expression difference from samples of the two groups are screened out based on results of statistical analyses;
S303, verifying the genes with the greatest expression difference, where the gene with the largest expression difference is validated using a real-time quantitative polymerase chain reaction (qPCR) method, and the expression difference of the gene in the samples of the two groups is further confirmed by quantitative analysis of gene expression levels; and
S304, conducting database querying and literature research, where public databases of systems of Genecards, The Human ProteinAtlas and National Center for Biotechnology Information are used to query relevant information about the C10ORF10 gene, and to obtain a mechanism of the gene in a function of the autolysosome.

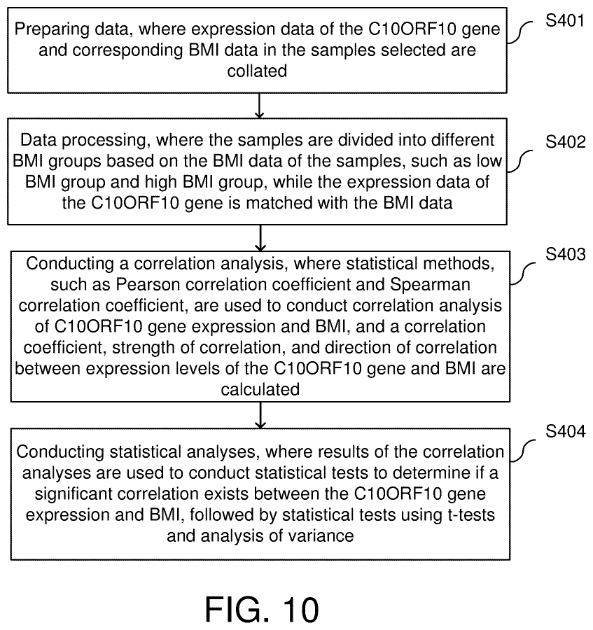

As shown in FIG. 10, specific steps of carrying out correlation analysis of a differential gene C10ORF10 with BMI in subcutaneous and visceral adipose tissues of people with different BMIs in the step 4 include:
S401, preparing data, where expression data of the C10ORF10 gene and corresponding BMI data in the samples selected are collated;
S402, data processing, where the samples are divided into different BMI groups based on the BMI data of the samples, such as low BMI group and high BMI group, while the expression data of the C10ORF10 gene is matched with the BMI data;
it also includes: S403, conducting a correlation analysis, where statistical methods, such as Pearson correlation coefficient and Spearman correlation coefficient, are used to conduct correlation analysis of C10ORF10 gene expression and BMI, and a correlation coefficient, strength of correlation, and direction of correlation between expression levels of the C10ORF10 gene and BMI are calculated;
it also includes: S404, conducting statistical analyses, where results of the correlation analyses are used to conduct statistical tests to determine if a significant correlation exists between the C10ORF10 gene expression and BMI, followed by statistical tests using t-tests and analysis of variance.

### Embodiment 2

A screening method of an autolysosome gene C10ORF10 for regulating the fat function of obese patients, including:
step 1, determining autolysosome genes, including 323 autolysosome genes as shown in FIG. 1A and FIG. 1B; collecting subcutaneous and visceral adipose tissues from people with different metabolic conditions and different BMIs, followed by transcriptome sequencing analysis;
step 2, dividing specimens from people with normal metabolism into two groups of BMI<25 (kg/m²) and BMI>30 (kg/m²), comparing the two groups in terms of expression differences of autolysosome-related genes in subcutaneous and visceral adipose tissues, screening out differential genes, followed by comparison of expressions of the differential genes in adipose tissues of the two groups in systems of Genecards, The Human ProteinAtlas, and the National Center for Biotechnology Information, selecting differential genes with high expression for analysis of a following step;
step 3, analyzing and observing the subcutaneous and visceral adipose tissues, comparing several autolysosome-related genes in terms of differential expressions existed in the two groups of BMI<25 (kg/m²) and BMI>30 (kg/m²), where a gene with a greatest expression difference is C10ORF10;
step 4, carrying out correlation analysis of a differential gene C10ORF10 with BMI in subcutaneous and visceral adipose tissues of people with different BMIs, where expression differences of C10ORF10 mRNA in adipose tissues of patients with different BMIs and correlation with BMI are firstly observed, and highly expressed C10ORF10 mRNA is observed in subcutaneous adipose tissues of patients with a BMI of >30 (kg/m²) (FIG. 3A and FIG. 3B) according to collected and analyzed RNA-seq results of human adipose tissues, suggesting a significant correlation with BMI (as shown in FIG. 4A and FIG. 4B);
step 5, conducting correlation analysis of differential genes with blood glucose, blood lipids, blood pressure and blood uric acid in obese patients with different metabolic conditions, including high uric acid, high blood lipids, high blood glucose, and high blood pressure; correlations of baseline C10ORF10 mRNA with baseline and post-follow-up reductions in blood glucose and HbA1c are observed in patients undergoing bariatric surgery, where C10ORF10 mRNA levels are observed to be positively correlated not only with postprandial glucose and HbA1c of baseline, but also with decreases in both postprandial glucose and HbA1c of post-follow-up (FIG. 5A-FIG. 5D), indicating that C10ORF10 mRNA is also involved in glucose regulation before and after weight-loss surgery in obese patients; and
step 6, constructing mice with conditional knockout or overexpression of the differential gene C10ORF10 in adipose tissue and performing related studies, including overall phenotypic observations, adiposity content and morphological observations in mice, adipogenic differentiation and tissue development, lipolysis, adipose tissue inflammation and overall inflammation, browning of subcutaneous fat, ectopic deposition of lipids in liver and skeletal muscles.

The above embodiments are only used to illustrate the present application and are not intended to limit the technical schemes described in the present application, although the present specification has been described in detail with reference to the above embodiments of the present application, the present application is not limited to the above specific embodiments, and therefore any modification or substitution of the present application, and all technical schemes and their improvements that do not deviate from the spirit and scope of the application are covered by the scope of the claims of the present application.

What is claimed is:

1. A screening method of an autolysosome gene C10ORF10 for regulating a fat function of obese patients, comprising:
determining autolysosome genes firstly, collecting subcutaneous and visceral adipose tissues from people with different metabolic conditions and different body mass indexes (BMIs), followed by transcriptome sequencing analysis;
dividing specimens from people with normal metabolism into two groups of BMI<25 kilogram per square meter kg/m² and BMI>30 kg/m², comparing the two groups in terms of expression differences of autolysosome-related genes in subcutaneous and visceral adipose tissues, screening out differential genes, followed by comparing expressions of the differential genes in adipose tissues of the two groups in systems of Genecards, The Human Protein Atlas, and the National Center for Biotechnology Information, and selecting differential genes with high expression for analysis; and
analyzing and observing the subcutaneous and visceral adipose tissues, comparing several autolysosome-related genes in terms of differential expressions existed in the two groups of BMI<25 kg/m² and BMI>30 kg/m², wherein a gene with a greatest expression difference is C10ORF10.

2. The screening method of the autolysosome gene C10ORF10 for regulating the fat function of obese patients according to claim 1, further comprising carrying out correlation analysis of a differential gene C10ORF10 with BMI in subcutaneous and visceral adipose tissues of people with different BMIs, wherein expression differences of C10ORF10 messenger ribonucleic acid (mRNA) in adipose tissues of patients with different BMIs and correlation with BMI are firstly observed, and highly expressed C10ORF10 mRNA is observed in subcutaneous adipose tissues of patients with a BMI of >30 kg/m² according to collected and analyzed ribonucleic acid (RNA)-seq results of human adipose tissues, suggesting a significant correlation with BMI.

3. The screening method of the autolysosome gene C10ORF10 for regulating the fat function of obese patients according to claim 2, further comprising conducting correlation analysis of differential genes with blood glucose, blood lipids, blood pressure and blood uric acid in obese patients with different metabolic conditions, comprising high uric acid, high blood lipids, high blood glucose, and high blood pressure; correlations of baseline C10ORF10 mRNA with baseline and post-follow-up reductions in blood glucose and HbA1c are observed in patients undergoing bariatric surgery, wherein C10ORF10 mRNA levels are observed to be positively correlated not only with postprandial glucose and HbA1c of baseline, but also with decreases in both postprandial glucose and HbA1c of post-follow-up, indicating C10ORF10 mRNA is also involved in glucose regulation before and after weight-loss surgery in the obese patients.

4. The screening method of the autolysosome gene C10ORF10 for regulating the fat function of obese patients according to claim 3, further comprising constructing mice with conditional knockout or overexpression of the differential gene C10ORF10 in adipose tissue and performing related studies, comprising overall phenotypic observations, adiposity content and morphological observations in mice, adipogenic differentiation and tissue development, lipolysis, adipose tissue inflammation and overall inflammation, browning of subcutaneous fat, ectopic deposition of lipids in liver and skeletal muscles.

5. The screening method of the autolysosome gene C10ORF10 for regulating the fat function of obese patients according to claim 4, wherein determining autolysosome genes comprises:
determining autolysosome genes, and identifying a list of autolysosome-related genes through literature research and database querying;
collecting samples, wherein obese patients with different metabolic conditions and different BMIs are selected as study objects and samples are collected from subcutaneous and visceral adipose tissues of the study objects, with a collection process following ethical regulations and relevant research ethics approvals obtained; and
extracting total RNA, wherein total RNA is extracted from subcutaneous and visceral adipose tissue samples using a method of RNA extraction kit according to instructions, and high quality of RNA is ensured.

6. The screening method of the autolysosome gene C10ORF10 for regulating the fat function of obese patients according to claim 5, wherein collecting subcutaneous and visceral adipose tissues from people with different metabolic conditions and different BMIs, followed by transcriptome sequencing analysis comprises:
preparing a library, wherein an RNA sample is used for library preparation according to a transcriptome sequencing methodology, typically comprising steps of RNA reverse transcription, cDNA synthesis, and library construction;
sequencing, wherein the library is subjected to high-throughput sequencing, either by an RNA-seq method for transcriptome sequencing, or by sequencing using an Illumina platform; and
analyzing data, wherein the data obtained from sequencing are subjected to pre-processing steps of quality control, removal of low-quality data and filtering of low-expressed genes, followed by comparative, quantitative and differential expression analyses of transcriptome data using bioinformatics analysis tools.

7. The screening method of the autolysosome gene C10ORF10 for regulating the fat function of obese patients according to claim 1, wherein analyzing and observing the subcutaneous and visceral adipose tissues, comparing several autolysosome-related genes in terms of differential expressions existing in the two groups of BMI<25 kg/m² and BMI>30 kg/m² comprises:
analyzing differential genes, wherein the expression differences of autolysosome-related genes in subcutaneous and visceral adipose tissues of the two groups of BMI<25 kg/m² and BMI>30 kg/m² are compared based on results of transcriptome sequencing analyses, and a statistical method is available to conduct differential gene analyses;
determining the gene with the greatest expression difference, wherein the gene with a largest expression difference is identified from the differential genes, and the autolysosome-related genes with a most significant expression difference from samples of the two groups are screened out based on results of statistical analyses;
verifying the genes with the greatest expression difference, wherein the gene with the largest expression difference is validated using a real-time quantitative polymerase chain reaction (qPCR) method, and the expression difference of the gene in the samples of the two groups is further confirmed by quantitative analysis of gene expression levels; and
conducting database querying and literature research, wherein public databases of systems of Genecards, The Human Protein Atlas and National Center for Biotechnology Information are used to query relevant information about the C10ORF10 gene, and to obtain a mechanism of the gene in a function of the autolysosome.

8. The screening method of the autolysosome gene C10ORF10 for regulating the fat function of obese patients according to claim 7, wherein carrying out correlation analysis of a differential gene C10ORF10 with BMI in subcutaneous and visceral adipose tissues of people with different BMIs comprises:
preparing data, wherein expression data of the C10ORF10 gene and corresponding BMI data in the samples selected are collated; and
data processing, wherein the samples are divided into different BMI groups based on the BMI data of the samples, such as low BMI group and high BMI group, while the expression data of the C10ORF10 gene is matched with the BMI data.

9. The screening method of the autolysosome gene C10ORF10 for regulating the fat function of obese patients according to claim 8, further comprising conducting a correlation analysis, wherein statistical methods involving Pearson correlation coefficient and Spearman correlation coefficient are used to conduct correlation analysis of C10ORF10 gene expression and BMI, and a correlation coefficient, strength of correlation, and direction of correlation between expression levels of the C10ORF10 gene and BMI are calculated.

10. The screening method of the autolysosome gene C10ORF10 for regulating the fat function of obese patients according to claim 9, further comprising: conducting statistical analyses, wherein results of the correlation analyses are used to conduct statistical tests to determine if a significant correlation exists between the C10ORF10 gene expression and BMI, followed by statistical tests using t-tests and analysis of variance.
